(19) United States
(12) Patent Application Publication
(10) Pub. No.: US 2021/0130763 A1
(43) Pub. Date: May 6, 2021

(54) DEVICE AND METHOD FOR STERILE SAMPLE-TAKING

(71) Applicant: bbi-biotech GmbH, Berlin (DE)

(72) Inventors: Bernd-Ulrich Wilhelm, Petershagen (DE); Jan Millauer, Berlin (DE); Ronnie Wilhelm, Berlin (DE)

(21) Appl. No.: 15/733,634

(22) PCT Filed: Mar. 20, 2019
(86) PCT No.: PCT/EP2019/057000

§ 371 Date: Jan. 14, 2021

(30) Foreign Application Priority Data
Mar. 23, 2018 (DE) 10 2018 204 477.5

(51) Int. Cl. C12M 1/26; C12M 1/12; C12M 1/36; C12M 1/00; C12M 1/06

(52) U.S. Cl. CPC C12M 33/12; C12M 37/02; C12M 27/02; C12M 29/00; C12M 41/48

(57) ABSTRACT

The invention relates to a device for sampling from bioreactors, comprising a rising line (1) connectable to a bioreactor, a tap line (2), and a withdrawal line (3), which are connected to a T-like connection. The withdrawal line (3) comprises a first shut-off valve (4), the tap line (2) is connected to a positive pressure source (5), and the withdrawal line (3) is connected to a pump (7). In the device for sampling, the positive pressure source (5), the pump (7), and the first shut-off valve (4) are controllable in an automated manner via a control unit (13). The invention furthermore relates to a method for using such a device for sampling.

The application relates to a device for sampling, and to a method for using the device.

Devices for taking samples from culture solutions, such as microorganisms or cells, for example from bioreactors, such as autoclavable culture vessels, fermenters or other cultivation systems, are known from the prior art. EP 2305790 shows such a device, wherein a sample can be taken manually from autoclavable culture vessels using a sterile syringe having a so-called Luer lock cone, via a connection. The device comprises an automatic valve, which automatically opens when the syringe is attached and automatically closes when the syringe is detached. The device is characterized in that a check valve closing towards the automatic valve is provided between the automatic valve and the connection. The device from EP 2305790 furthermore shows a tap line which includes an air filter and a check valve and is connected to a through-conduit between the automatic valve and the connection via a T piece. For rinsing the device, a syringe is connected to the tap line according to EP 2305790, and the plunger is squeezed out as often as is needed to completely force the culture solution from the lines into the culture vessel.

The operation by hand requires regular intervention from outside. Especially during longer experiments, manual operation is complex and cost-intensive. Furthermore, faulty operation may result in contamination of the bioreactor or of the sample.

It is the object of the present application to develop a device for sampling in which contamination of the sample or of the bioreactor is avoided, and which is designed so as to be usable fully automatically.

The object is achieved by a device having the features of claim 1.

Advantageous embodiments will be apparent from the dependent claims.

A device within the meaning of the present application comprises a rising line connectable to a bioreactor, a tap line, and a withdrawal line. The rising line, the tap line, and the withdrawal line are connected by means of a T-like connection.

The withdrawal line comprises a first shut-off valve. The tap line is optionally connected, or connectable, to a positive pressure source, and the withdrawal line is optionally connected, or connectable, to a pump.

The device furthermore comprises a control unit for controlling the positive pressure source, the pump, and the first shut-off valve.

As a result of this configuration, the positive pressure source, the pump, and the first shut-off valve can be controlled automatically in that the individual components are activated or actuated by the control unit in an established order. In this way, it is possible to take samples or carry out measurements in the delivered medium without human intervention. For this purpose, the valves, the positive pressure source, and the pump, or further possible components of the device, can comprise corresponding actuators and/or motors and/or be connected thereto, so as to enable such an activation or actuation triggered by the control unit. In the case of valves, activation or actuation means opening and closing, for example. In the case of the pump and the positive pressure source, this shall be understood to mean switching on and off. In general, this may, for example, be understood to mean a change in an operating state, which in the present case can preferably be carried out without human intervention and in a manner controlled by the control unit.

This in particular simplifies the process of carrying out multiple samplings and/or measurements over an extended period of time. It is then not necessary, for example, for a person to be present during the sampling process so as to trigger or carry out this process manually.

A T-like connection, via which the rising line, the tap line, and the withdrawal line are connected to one another, shall be understood to mean all connections that allow three lines to be connected, that is, for example, also connections that, in terms of the geometry thereof, represent more of a Y-like connection. The use of a standardized and/or commercially available T piece represents a possible embodiment, but is not mandatory within the meaning of the application.

The lines can be designed as flexible tubing, for example.

The positive pressure source can be designed as a syringe or in a syringe-like manner, for example. It can therefore be a receptacle comprising a movable stud, for example, which can be lowered in a manner similar to a syringe, so as to generate positive pressure. The positive pressure source can also be designed as a process gas source or as a sterile gas source, for example as a gas line or a pressurized receptacle. The process gas is preferably a gas that does not influence, or does not negatively influence, the biological process inside the reactor. For example, nitrogen can be used as the positive pressure gas. The process gas source or sterile gas source can be provided in the form of a correspondingly connected, for example replaceable, gas tank, or in the form of a gas line. The positive pressure source can also comprise a pump for generating pressure. The positive pressure source can preferably be controlled in an automated manner, thus comprising a mechanism that provides the positive pressure in an electronically activatable manner, without requiring human intervention.

The pump that is connected to the withdrawal line can, for example, be configured as a tubing pump, such as a peristaltic pump or roller pump. For this purpose, the withdrawal line is preferably designed as flexible tubing, at least in the region of the pump. The pump is used to generate negative pressure. As a result of the negative pressure, the sample can be drawn out of the culture vessel connectable to the device, for example a sample vessel connectable to the withdrawal line. The sample vessel is made of plastic or stainless steel, for example.

By arranging the first shut-off valve in the withdrawal line, the withdrawal line can be closed, and the rising line can be blown out by means of the positive pressure source connectable to the tap line. The delivered medium can thus be forced out of the rising line. In contrast to configurations in which the withdrawal line is not closable, the line volume to be blown out, which has to be purged by means of the positive pressure source, can be reduced here, enabling simpler, faster, and more reliable cleaning or rinsing. In addition, a blow-out process decreases the risk of contamination since gas, process gas, outside air, sample material or foreign objects cannot reach the lines closed by the first shut-off valve.

In one embodiment, the first shut-off valve and additional valves that can be installed in the device according to the application are designed as electrically or pneumatically controllable valves so as to allow the sampling process to be automated. The valves are preferably designed or arranged so as not to come in contact with media, for example as pinch valves.

As mentioned, the described device allows automated sampling, for example in that steps in which the pump or the positive pressure source is switched on or off, and the first shut-off valve and possible further shut-off valves are opened or closed, are carried out in a pre-programmed manner. These steps can be carried out and repeated, for example, in a time-controlled manner, at pre-programmed intervals. Possible further components of the device can likewise be in communication with the control unit and receive control signals from the control unit and, in some embodiments, also transmit signals or data to the control unit. Possible configurations that comprise further components as well as possible interactions between the components and the control unit are described hereafter.

A system according to the present application comprises, for example, a device as described in the application, or a device according to any one of the patent claims, and a bioreactor connected to the rising line. The bioreactor can, for example, be an autoclavable vessel or a vessel sterilizable by way of steam or sterilizable in another manner.

In one specific embodiment, the tap line, between the pump and the T-like connection, comprises a sterile filter so that the lines continue to be kept sterile when using a non-sterile positive pressure source. So as to avoid blocking of the sterile filter or of the positive pressure source, an optional check valve can furthermore be provided in the tap line. In a combination comprising a sterile filter, this is arranged between the sterile filter and the T-like connection so as to be open for fluid flow in the direction of the T-like connection, and closed in the opposite direction.

A further optional check valve can be arranged in the withdrawal line. It is oriented in such a way that fluid flow is made possible from the T-like connection in the direction of the pump, and flow is prevented in the opposite direction.

The device can furthermore comprise a second shut-off valve, which is arranged in the rising line and preferably designed as an electrically controllable valve, and which is connectable to the control unit so that functions relating to this second shut-off valve are also automatable.

The device can furthermore comprise one or more sensor units, for example arranged in the rising line and/or in the withdrawal line. Each of the one or more sensor units can comprise one or more sensors, which are used to record one or more measurement values describing a state or a property of the sample. Furthermore, a sensor unit can also be arranged in the bioreactor so as to measure measurement values of the sample still present in the bioreactor. For example, a thermometer arranged in the bioreactor can continuously monitor the temperature in the reactor.

In one embodiment, at least one of the sensor units is connected to the control unit and configured to transmit measurement values to the control unit and/or configured to be controlled by the control unit.

Sensors connected to the control unit can trigger a pre-programmed sequence of steps for sampling, or for further measurements, by way of a signal transmitted to the control unit.

A sampling or a measurement can be triggered, for example, by a value measured by the sensor which exceeds or fails to meet a preset limit value, for example. The sample is thus taken when a certain event occurs. The pre-programmed steps for sampling can thus, within this meaning, be event-controlled, that is, be triggered by the measurement of a certain value by a sensor. The measurement value is transmitted from the sensor to the control unit in the process, which then, in turn, provides the corresponding signals for sampling to the units required for sampling, such as pumps, shut-off valves, and positive pressure sources.

In addition to the event-controlled sampling process, there is the option of time-controlled sampling, as described above, which is carried out automatically by the control unit at times established in advance, wherein, for example, a starting point in time and time intervals are established by a user. Time-controlled and event-controlled sampling can be used as alternatives or in combination. Likewise, it is possible for the time control and the event control to be in interaction with one another and to represent boundary conditions for one another. For example, a sampling process can be carried out at a certain time on condition that a certain sensor signal is present, or a sampling process can be carried out at a certain sensor signal on condition that an established time period has passed since the last sampling.

Several additional possible examples for sensors and the arrangement or use thereof are mentioned hereafter, wherein a person skilled in the art can also select different sensors or arrangements, depending on the sample or application.

Different physical, chemical or biological sensors may be used as sensors.

Examples include mechanical, electrical, optical or micro-electrochemical sensors, electrochemical sensors, enzymatic sensors, or bioarray or biochip sensors.

For example, optical sensors can be used fora spectral analysis or for measuring optical density. Furthermore, chemical sensors can be used for pH measurement or for oxygen measurement. Enzymatic sensors can be used to detect the reaction of an enzyme proportional to the concentration of a substrate to be determined, such as glucose, lactate, glutamate, glutamines, and the like.

Depending on which measured variable is to be recorded, the sensors can be designed as contactless sensors or sensors that come in contact with media.

It is a further object of the invention to arrange and use the sensors in such a way that measurement-induced influence or contamination of the sample is avoided to the extent possible, or only occurs when this is unavoidable. Such a problem arises in particular in connection with sensors that come in contact with media.

For example, a measurement by means of a sensor coming in contact with media in general prevents the sample from being pumped back into the bioreactor afterwards since it is consumed by the measurement, for example as a result of a chemical reaction or a biological process, or altered in such a way that it would contaminate the remaining material in the bioreactor.

Using different configurations shown here, it can be avoided that sample material is unnecessarily wasted by such measurements because more material than necessary is withdrawn, or because the material is withdrawn too soon. In one embodiment of such a measurement with media contact, a contactless measurement is thus provided upstream, which is suitable for determining whether the sample material present in the bioreactor has already reached a state in which the measurement with media contact is expedient. For example, an upstream optical analysis can be used to determine whether a reaction in the bioreactor has sufficiently progressed to carry out a measurement with media contact. Based on such an upstream contactless measurement, it can also be determined whether sampling is to take place. The contactless sensors arranged upstream in the line thus, for example, record measurement values that make it possible to decide whether a further measurement is carried out and/or a sample is taken.

In one possible design according to the application, a first sensor unit comprising a contactless sensor and a second sensor unit comprising a sensor coming in contact with media, for example, are thus consecutively arranged, wherein preferably at least one shut-off valve is arranged between the two sensor units. This is made possible by an upstream measurement by means of the contactless sensor, whereupon, based on the measurement values ascertained by the contactless sensor which are preferably transmitted to the control unit, preferably in an automated manner, a decision is made, for example by a comparison to user-defined values, as to whether the sample is either pumped back into the bioreactor, or whether the sample is pumped onward so as to be analyzed by the sensor coming in contact with media and/or to be withdrawn.

If a first sensor unit is present in the rising line, this may, for example, be located on the side of the second shut-off valve which faces the bioreactor. In one embodiment, this first sensor unit is only equipped with contactless sensors.

In different embodiments, it is also possible that only the first or only the second sensor unit is present.

The second sensor unit can be arranged between the first shut-off valve and the pump. The one or multiple sensors of the second sensor unit can be designed as sensors that come in contact with media or as contactless sensors.

After having been measured by a sensor of the first and/or second sensor units which comes in contact with media, the sample can then be discarded, stored, or be made available for other measuring processes. Pumping onward and/or pumping back is made possible by pumps and valve systems provided in the application.

For example, signal attenuation during absorption measurement, which is carried out by means of a sensor of the first sensor unit, may indicate an increase in biomass, that is, an increased cell count. Starting at a certain biomass, for example, a substrate (for example glucose) has to be delivered, the concentration of which is then measured by a measurement with media contact in an enzymatic sensor of the second sensor unit. The second measurement by way of the enzymatic sensor is expensive, and the number of measurement cycles of the sensor is typically limited. The measurement by way of the sensor of the second sensor unit thus does not commence until the sensor of the first sensor unit has indicated a sufficient cell concentration.

In one embodiment, the substrate concentration is to be kept constant, for example, despite an increase in the cell concentration, which can be ensured by way of automatic feedback to a possible substrate pump, which is likewise coupled to the control unit and controllable thereby. For this purpose, measurements of the substrate concentration by means of the first sensor unit are carried out at close intervals.

Another object of the invention relates to the practical handling of the sampling device. This also encompasses, for example, preparatory steps necessary to enable a safe and contamination-free reaction process, and safe and contamination-free sensor measurements and sampling processes.

In addition to the described arrangements of valves and sensors, safe and contamination-free handling by a device according to the application can furthermore be facilitated by one or more sterile couplings, which allow individual components or sections of the device to be sterilized and furthermore make it possible to replace sensors or other components, in some embodiments even during the reaction process.

For example, the sterile couplings are integrated into the lines, as described or shown hereafter or in the figures of the present application.

In particular the sterilization of the sensors of the sensor units which come in contact with media frequently presents a challenge for a person skilled in the art since these, due to the direct contact thereof with the sample, have to be sterile in some instances, but often cannot be sterilized together with the bioreactor, since they, for example, are not temperature-resistant and thus are not autoclavable or steam-sterilizable. While the bioreactor is preferably sterilized by autoclaving or steam sterilization, the sensors coming in contact with media, for example, are not suitable for these types of sterilization and instead have to be sterilized, for example, by way of gamma sterilization, electron beam sterilization, plasma sterilization, or by means of ethylene oxide sterilization. This may similarly apply to other components of the device, so that the solution to this problem described here can be analogously applied to additional components.

In some cases, the sensors coming in contact with media can also be unsterile and, according to the application, sterile couplings are arranged so as to be separate, or separable, from a sterile portion of the device.

In contrast to the sensors coming in contact with media, the contactless sensors are typically easy to integrate into the device since they are not in direct contact with the sample and can be removed prior to sterilization. In terms of the arrangement of the contactless sensors, additional options arise accordingly, which are utilized in the described configurations. The same circumstances can apply, for example, to the shut-off valves that are used if these are configured as pinch valves, for example.

The sterile couplings that are used comprise two joinable unions, for example. These are provided at two line ends, which are to be connectable to one another. So as to connect the line ends, the unions can be attached to one another in a fluid-tight manner, and be pulled apart again so as to disconnect the line ends, and when separated, the disconnected line ends are preferably sealed by the sterile coupling. In one embodiment, the line ends are sealed in a fluid-tight manner by the sterile coupling, at least before they are attached to one another for the first time.

For example, the unions can be designed as two complementary plastic unions, wherein each of the unions can comprise a diaphragm, which seals the particular line end and can be pulled out after the two plastic unions have been fastened or coupled so as to release a fluid path through the sterile coupling. Additionally, needle-diaphragm coupling systems may be used, for example.

One specific embodiment provides for the use of gamma-sterilizable disposable couplings made of plastic. Likewise, the use of steel-steel or steel-plastic couplings is provided, wherein in the latter case a steel coupling union is preferably arranged on the side of the bioreactor, and a plastic coupling union is preferably arranged on the side of the sensor.

The one or more sterile couplings can, for example, be arranged in the rising line and/or in the withdrawal line and allow a line section and the components present thereon to be disconnected from and connected is the remaining device.

For example, in a device in which the first sensor unit is present in the rising line and/or the second sensor unit is present in the withdrawal line, and at least one of the sensor units comprises a sensor coming in contact with media, a sterile coupling can be arranged in the rising line as a first component downstream of the bioreactor. In this possible embodiment, the sampling device, including all shut-off valves, check valves, sensor units, and potential further components, as well as a first coupling union, can thus be disconnected from the bioreactor. A section of the rising line as well as a second coupling union remain at the bioreactor itself.

The bioreactor, including the section of the rising line and the second coupling union, are autoclaved before the coupling union is coupled. The remaining portion of the device can then be sterilized, for example, by means of gamma sterilization or by means of another method for which the sensors that are used are designed.

If a sensor coming in contact with media is only present in the withdrawal line in the second sensor unit, while the first sensor only comprises contactless sensors, or if the first sensor unit is completely dispensed with, a sterile coupling can, for example, be arranged between the first shut-off valve and the second sensor unit. The bioreactor and the first and second shut-off valves are then located on the side of the first coupling union, and the second sensor unit, together with potential further components, such as rinsing lines and/or mixing chambers, are located on the side of the second coupling union.

Such a configuration allows the section including the first coupling union and encompassing the bioreactor to be sterilized in a preparatory manner. This section typically also comprises the tap line including the check valve and the sterile filter. The section on the side of the second coupling union which includes the second sensor unit comprising the sensor coming in contact with media may also be unsterile, or may be gamma-sterilized, for example. The sensor coming in contact with media may be a glucose sensor or an enzymatic sensor, for example. The configuration can prevent a sample that passes the second shut-off valve, and thus necessarily reaches the possibly unsterile region, from contaminating the bioreactor. This arrangement of the sterile coupling makes it possible to replace the second sensor unit during ongoing operation by disconnecting the sterile coupling. The sterile portion of the device which includes the bioreactor and the first and second shut-off valves remains unaffected by the replacement. In this way, it is possible to continue to carry out contactless measurements by way of the possible first sensor unit even while the second sensor unit is decoupled. This solves a problem that relates to the long-term operation of such bioreactors. The run time of enzymatic sensors, for example, is typically limited to several weeks. The device described here enables measuring over a longer period since the sensor, having reached the end of the service life thereof, can be decoupled during operation, and a new sensor can then be coupled again.

In one specific embodiment, a system within the meaning of the application is designed in such a way that the device for sampling is integrated into the bioreactor. For example, the device for sampling can be installed into an outer plate, such as a front plate or side plate of the bioreactor. Such an arrangement is particularly space-saving and makes it possible to provide the sampling system in a simple manner. In one embodiment, the user is spared complicated mounting, assembling of tubing, and checking of connections. He or she only has to mount the outer plate equipped with the sampling system to the bioreactor, for example.

Such a compact arrangement or integration allows a rapid analysis, wherein additionally only a small volume of media has to be withdrawn from the bioreactor in each case for a sensor measurement by means of a sensor of the first and/or second sensor units.

The use of a system according to the application comprises, for example, at least one step for sampling, in which the first and second shut-off valves are open, and the sample is drawn from the bioreactor by means of the pump. Furthermore, the use can comprise at least one step for blowing out the withdrawal line, wherein positive pressure is applied to the tap line by means of the positive pressure source, while the pump is being operated, and the first shut-off valve is open and the second shut-off valve is closed. Furthermore, the use can comprise at least one step for blowing out the rising line by applying positive pressure by means of the positive pressure source, while the second shut-off valve is open and the first shut-off valve is closed.

The durations of the individual steps can be programmed in advance. Depending on the duration, a certain action is achieved. For example, a larger sample volume is withdrawn if the step for sampling lasts longer. The steps for blowing out can, for example, last so long that during the blow-out period, corresponding to the amount of positive pressure provided by the positive pressure source, the amount of air introduced into the pipe to be blown out approximately corresponds to that which the pipe to be blown out is able to hold, so as to ensure, on the one hand, that the pipe is purged completely, without building unnecessarily high pressure and/or introducing excessive amounts of process gas, or of another gas used for blowing out, into the bioreactor or into the sample vessel, for example.

Using such a method, organisms remaining in the rising line are forced back into the bioreactor, and organisms remaining in the withdrawal line are recirculated to the sample. In this way, dead volume in all lines can essentially be reduced to zero since no medium with cells is lost, but all the material can either be recirculated to the sample or to the bioreactor. A minimum sample volume can thus be reduced to a few milliliters, for example to 5 milliliters.

The described steps can be carried once or multiple times during a single sampling process. The rising line is preferably purged prior to and after sampling. Further steps for starting up the pump and/or for building pressure or reducing pressure are preferably carried out.

The steps can be carried out manually or be programmed and carried out fully automatically. The execution of the steps can, for example, be triggered automatically in a time-controlled or event-controlled manner, for example based on a value measured by a sensor. In the case of a time-controlled sequence, the selection can be limited to a starting point in time, an interval between samplings, and a number of samplings.

If the sampling is to be triggered in an event-controlled manner by a value measured by a sensor arranged in the rising line, the following method can, for example, be employed: At a particular point in time, for example a point in time programmed in advance or triggered by an input by the user, negative pressure is generated by means of the pump, with the shut-off valves open. This negative pressure, however, is only sufficient to draw organisms into the rising line and reach the level of the sensor, but preferably not the T-like connection or the withdrawal line. The sensor then carries out a measurement. Based on the result of the measurement, which can be above or below an established limit value, for example, a sampling process is then either carried out or is not carried out. If the measurement value is such that sampling is desired, negative pressure continues to be generated in the pump, and organisms are conducted through the withdrawal line into a sample vessel, and thereafter the lines are blown out as described above. If the measurement value is such that no sampling is to be carried out at the point in time, for example since the process in the bioreactor has not yet sufficiently progressed, the first shut-off valve can automatically be closed again, and positive pressure can be provided by means of the positive pressure source, with the second shut-off valve open, and the organisms present in the rising line can be recirculated into the bioreactor. After an established time interval, a renewed measurement can then be carried out by means of the sensor in the rising line, as described above, and it can be decided as to whether or not the sample is to be taken. According to the application, different methods can be combined with one another for sampling, these being event-controlled and time-controlled.

Additional steps that can be carried out within the scope of a method according to the application are, for example, steps for preparing a measurement, such as rinsing steps in which a buffer solution is pumped into the tubing system, steps for the dilution or pressure filtration or other pretreatment of the sample. A mixing chamber can furthermore be integrated into the line system, preferably downstream of the first shut-off valve, for pretreating the sample.

The control unit can, for example, be part of a bioreactor control system or be integrated therein. In this way, the valves, the pump, and the positive pressure source can, for example, be connected to the bioreactor control system and be controllable by way of the bioreactor control system, for example via the control system of a xCUBIO reactor.

The device for sampling will be described in greater detail hereafter based on several exemplary embodiments.

In the drawings:

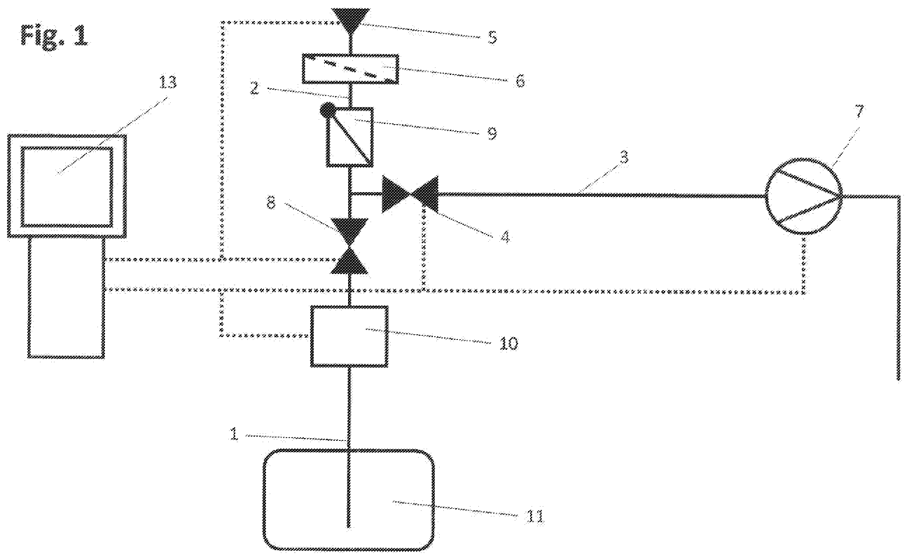

FIG. 1 shows a bioreactor system, a bioreactor, and a device for sampling comprising a first sensor system;

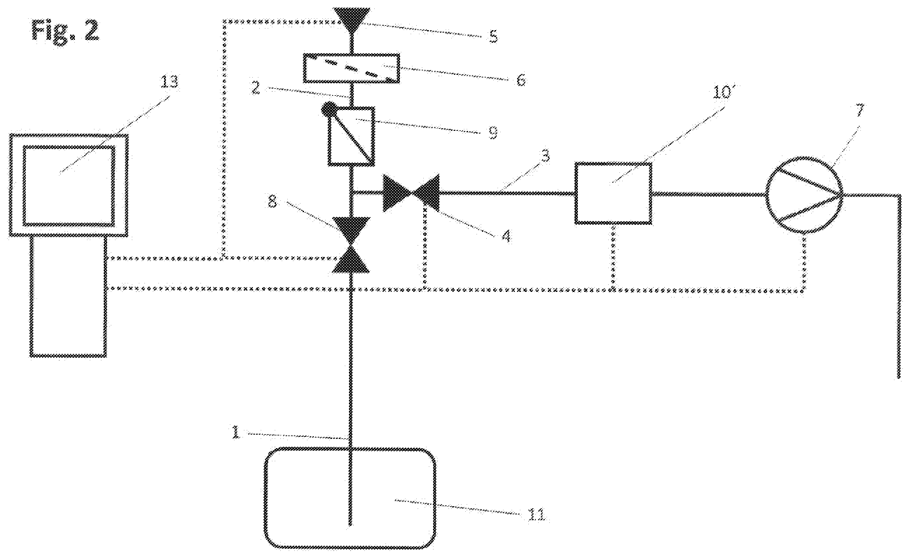

FIG. 2 shows the bioreactor system comprising a second sensor system;

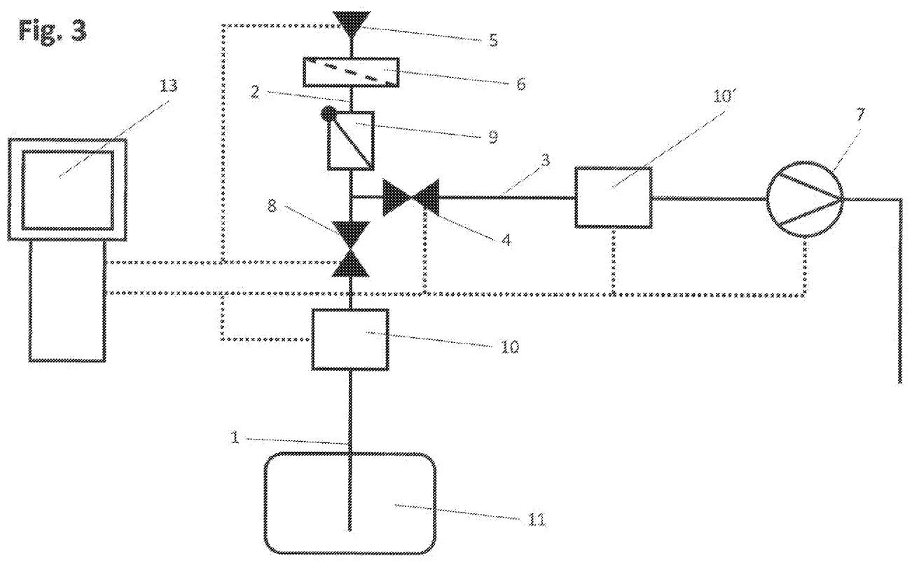

FIG. 3 shows the bioreactor system comprising a third sensor system;

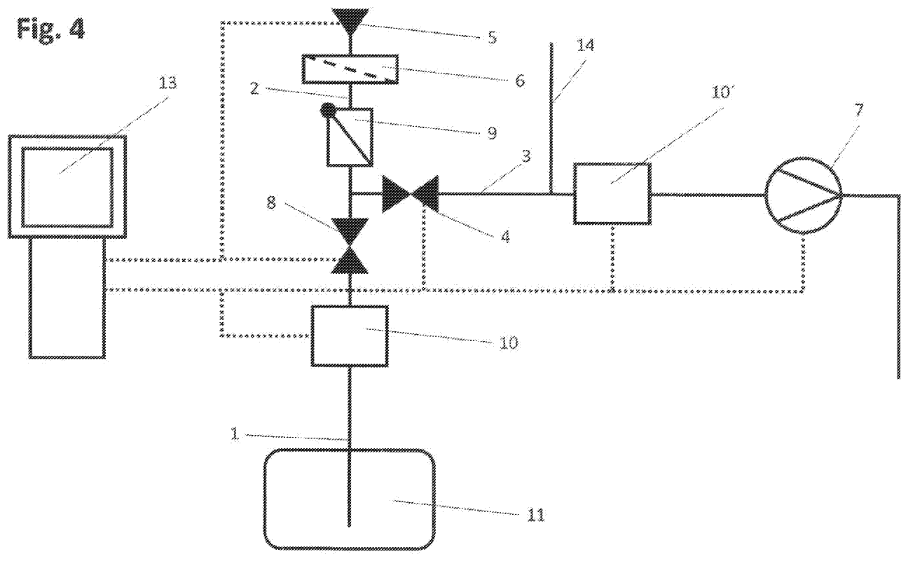

FIG. 4 shows the bioreactor system from FIG. 3, comprising a rinsing line;

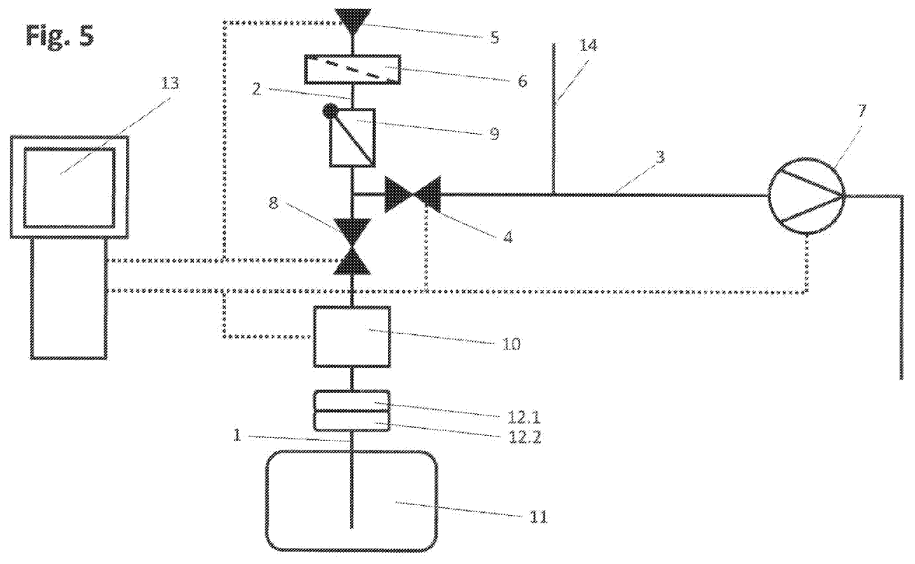

FIG. 5 shows the bioreactor system comprising the first sensor system and a sterile coupling;

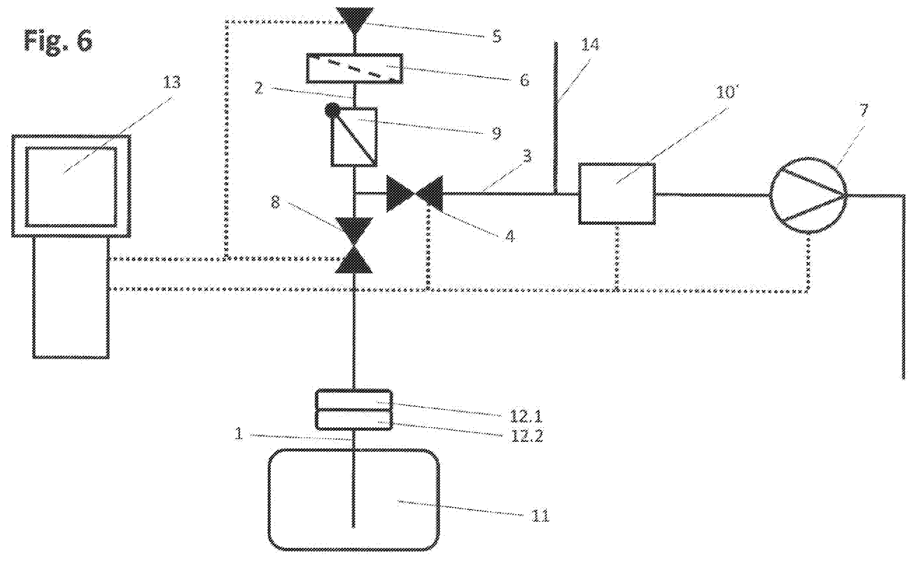

FIG. 6 shows the bioreactor system comprising the second sensor system and the sterile coupling;

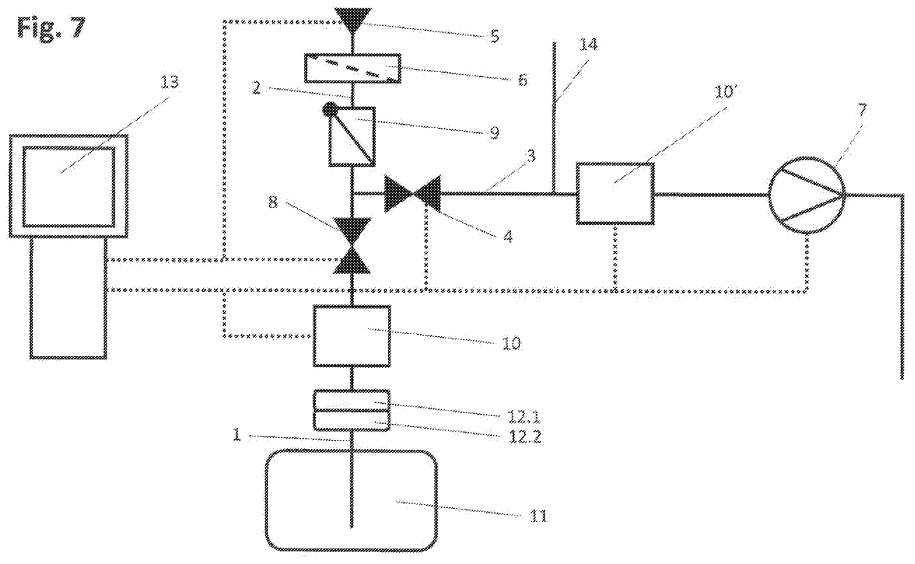

FIG. 7 shows the bioreactor system co rising the third sensor system and the sterile coupling;

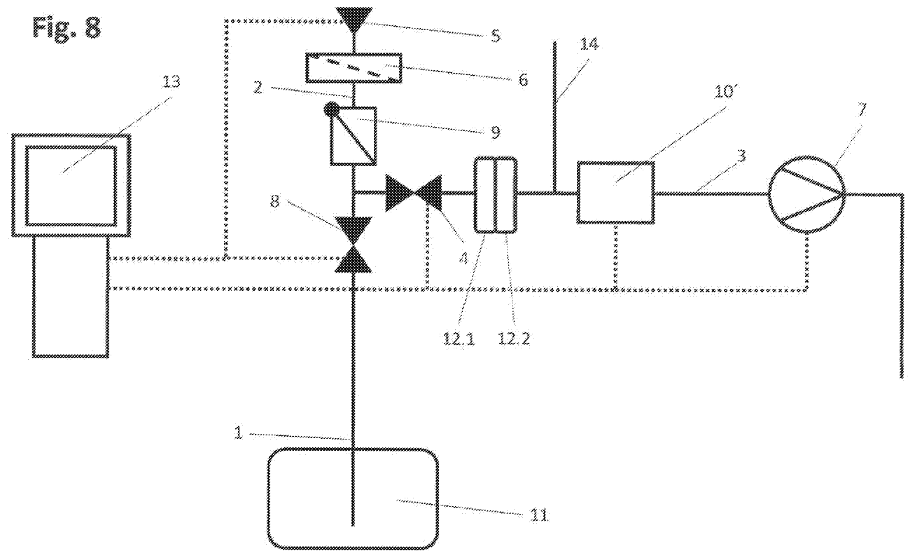

FIG. 8 shows the bioreactor system from FIG. 6, having an alternative arrangement of the sterile coupling;

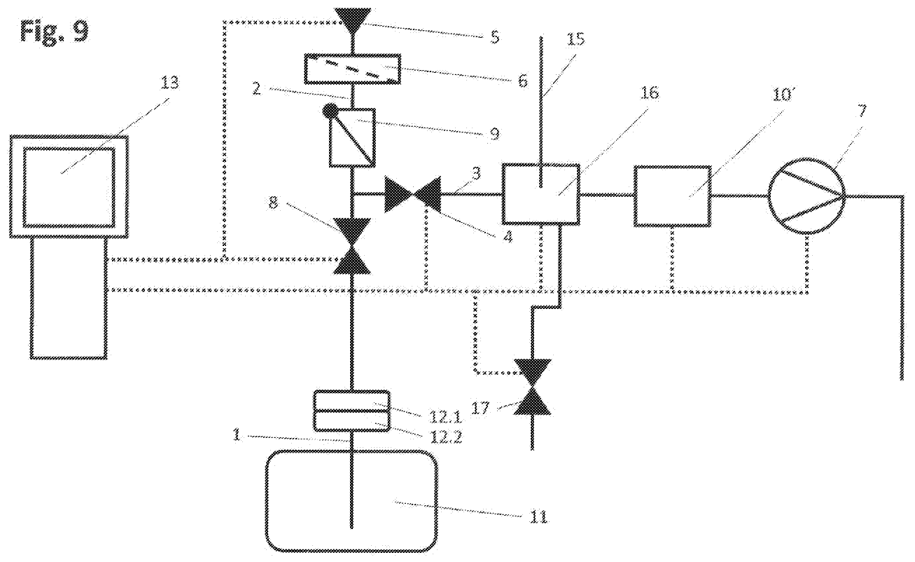

FIG. 9 shows the bioreactor system from FIG. 6, additionally comprising a mixing chamber; and

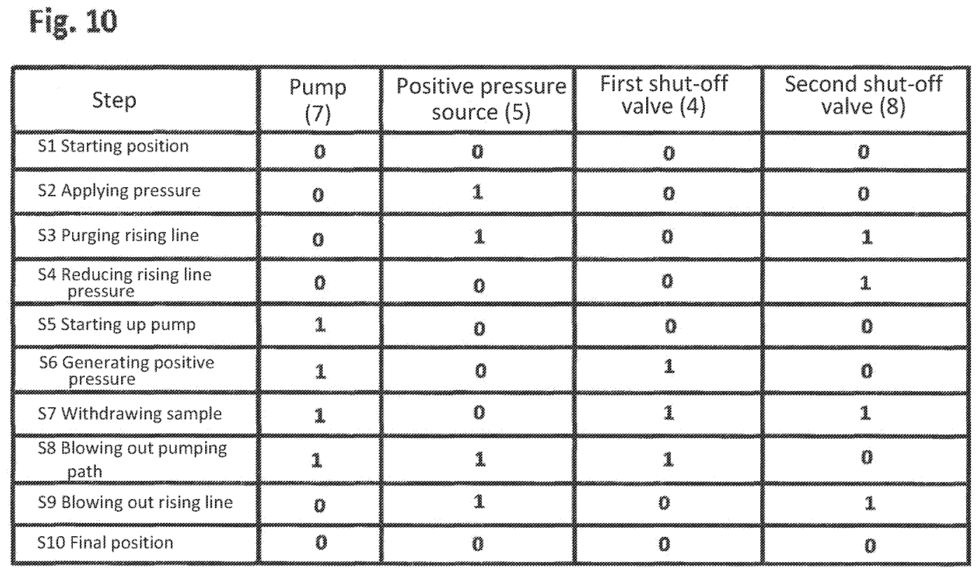

FIG. 10 shows a tabular presentation of a sequence of steps for use of the bioreactor system.

FIG. 1 shows a bioreactor system, wherein a bioreactor 11, which is designed as an autoclavable or as a steam-sterilizable vessel, for example, is connected to a rising line 1 of a device for sampling. On the side facing away from the bioreactor 11, the rising line 1 is connected in a T-like manner to a tap line 2 and a withdrawal line 3, for example by means of a T piece. The withdrawal line comprises a pump 7, for example a roller pump or a peristaltic pump, by which negative pressure can be generated in the withdrawal line 3. The withdrawal line 3 furthermore comprises a first shut-off valve 4. The rising line 1 comprises a second shut-off valve 8, which can be used to close the rising line 1. A first sensor unit 10, which is equipped with one or more sensors, is located on the side of the second shut-off valve 8 which faces the bioreactor 11. In this embodiment, the first sensor unit is preferably equipped with contactless sensors, for example with optical sensors. The device according to the application is not limited to the effect that one or more sensors have to be provided in a single first sensor unit 10. It is also possible to distribute multiple sensors among multiple sensor units. On the side facing away from the T-like connection, the tap line 2 comprises a positive pressure source 5 for generating positive pressure in the tap line 2. Such a positive pressure source 5 can be designed as a simple syringe or another gas source, such as a pressurized gas receptacle or a receptacle comprising a movable stud, which can be pressurized by lowering the stud, similarly to a syringe, preferably in an automatically controllable manner. The gas used for generating the positive pressure can, for example, be a process gas, nitrogen or air. It may stem from a sterile gas source, for example. The gas stemming from the positive pressure source 5 can be filtered by an additional possible sterile filter 6, which is arranged in the tap line, before it comes in contact with the organisms present in the bioreactor 11 or in the remaining lines 1, 2. A check valve 9, which is arranged in the tap line 2 on the side of the sterile filter 6 which faces the T-like connection, is provided for protecting the sterile filter 6. This check valve serves as an additional safety measure, but may also be dispensed with since negative pressure preferably does not arise in the tap line, which causes organisms to find their way into the tap line, but preferably positive pressure for blowing out is generated in the tap line 2, while negative pressure is preferably only generated in the withdrawal line 3. The withdrawal line 3 comprises a pump 7 for generating negative pressure in the withdrawal line 3, in particular in relation to the rising line 1 and in relation to the bioreactor 11. The withdrawal line 3 furthermore comprises a first shut-off valve 4. The withdrawal line 3 can optionally furthermore comprise a check valve, which is not shown and allows fluid to flow only from the T-like connection in the direction of the pump 7, but not in the opposite direction.

It is preferably possible to sterilize the lines together with the bioreactor 11. For example, the bioreactor 11 can be autoclaved together with the lines 1, 2, 3 designed as flexible tubing, wherein tubing ends are preferably kept closed so as to prevent contamination after sterilization. The sterile coupling 6 also helps to prevent the contamination. After the sterilization, the shut-off valves 4, 8 can be mounted. The first sensor unit 10 can also preferably be attached to the outside of the rising line 1 designed as flexible tubing and, for example, measure optical density through the tubing.

After sterilization, the device can be set up as described above, and a reaction can be started in the bioreactor.

The device according to the application can then be used to carry out different methods for sampling during the reaction, during which the individual elements of the device, such as the shut-off valves 4, 8, the pump 7, and the positive pressure source 5 are activated, or opened and/or closed, according to a particular pattern.

The pump 7, the positive pressure source 5, as well as the first and second shut-off valves 4, 8 can be activated via a control unit 13. The communication link between the individual components and the control unit 13 is indicated by dotted lines in the figure. For example, the control unit 13 can be programmed in such a way that the withdrawal process is carried out at established time intervals. For this purpose, the user can predefine, for example, a starting point in time, an interval between the samplings, and a number of samplings. However, the control unit 13 can also be in communication with the sensor 10, so that sampling by means of a pre-programmed sequence of steps, which is described in more detail hereafter, is triggered when a measurement value measured by the sensor of the first sensor unit 10 meets an established criterion. It is also possible that a sampling process that is intended at an established point in time is only carried out when the measured value meets such a criterion. In this way, it is possible, for example, to start a sampling process at an established point in time, wherein sample material is pumped from the bioreactor 11 into the rising line, at least up to the first sensor unit 10. Based on the measurement values recorded by the sensor of the first sensor unit 10, the sample is either pumped onward and withdrawn, or pumped back into the bioreactor.

In this way, it is made possible in particular to carry out multiple sampling processes or measurements distributed over a longer period of time, and to minimize the need for human interaction to the extent possible.

Possible steps for taking a sample or collecting measurement values, which are advantageous for the use of the shown sampling process, are described hereafter.

The execution of all steps is controlled via the control unit 13, which receives signals from the individual components or transmits signals to the individual components.

During a sampling process, sterile compressed air can be introduced into the tap line 2 in a preparatory manner by means of the positive pressure source, in conjunction with the sterile filter 6, wherein the first shut-off valve is preferably kept closed, while the second shut-off valve is opened. As a result, sample material remaining in the rising line 1 connected to the tap line 2 is forced back into the bioreactor. The second shut-off valve can then be closed.

Thereafter, the pump 7 is started up, wherein the first shut-off valve 4 is preferably initially kept closed. Thereafter the first shut-off valve 4 is opened, and the second shut-off valve 8 is kept closed, so as to generate negative pressure in the withdrawal line 3, or establish positive pressure in the bioreactor 11, and at least a portion of the rising line 1, compared to the withdrawal line 3. In a later step, the second shut-off valve 8 is then also opened, and during continued operation of the pump 7 an established amount of the organisms present in the bioreactor 11 is pumped or drawn out of the bioreactor 11 in to the rising line 1. These organisms pass the first sensor unit 10 arranged in the rising line 1 and are measured by means of the one sensor, or by means of at least one of the multiple sensors, of the first sensor unit 10, and the measurement values are transmitted to the control unit 13.

For example as a function of the measurement values thus recorded, which are, for example, compared to user-defined threshold values in the control unit 13, the organisms can then be forced back into the bioreactor 11 in that the first shut-off valve 4 is closed by the control unit 13 and the positive pressure source 5 is activated. After the sample has been forced back into the bioreactor, both shut-off valves 4, 8 can be closed. The steps that relate to forcing back the sample after the measurement are provided in the devices according to the application in which one or more sensors are arranged in the rising line 1. As is described hereafter in connection with other figures, not all devices provide sensors in the rising line 1, so that the steps for forcing back are typically not used in methods for using device having no sensors in the rising line 1.

As an alternative, it is possible to withdraw the sample, as a function of the result of the comparison of the measurement values to the threshold values, by pumping the sample, after it has passed the first sensor unit 10, into the withdrawal line 3 by continuing to operate the pump 7, with the shut-off valves 4, 8 open, and through the withdrawal line 3, for example into a sample vessel (not shown).

For rinsing the pumping path, the second shut-off valve 8 is then closed again, and positive pressure is generated in the tap line by the positive pressure source 5. The withdrawal line is blown out by simultaneously continuing to operate the pump 7 and keeping the first shut-off valve 4 open. Thereafter, the first shut-off valve 4 can be closed, and the second shut-off valve 8 can be opened, while keeping the positive pressure source 5 switched on. The pump 7 can be switched off in the process. In this way, the rising line 1 is blown out. The duration of this blowing-out step of the rising line 1 can be selected in such a way that only a small amount of gas is introduced into the bioreactor. So as to avoid contamination, the steps for blowing out the withdrawal line 3 and the rising line 1 can be carried out not only after, but also before a sampling process.

FIG. 2 shows the bioreactor system, wherein a second sensor unit 10′ is arranged in the withdrawal line 3, instead of the first sensor unit 10 shown in FIG. 1. The second sensor unit 10′ can comprise contactless sensors and/or sensors that come in contact with media, provided the sensors coming in contact with media are suitable to be sterilized together with the bioreactor.

In this configuration, the steps for sampling are carried out similarly to the manner described in connection with FIG. 1, wherein here, however, no option exists for pumping the sample back into the reactor after the measurement. As mentioned above, typically no steps for forcing the sample back are thus carried out. Instead, the sample is withdrawn as described above, wherein measurement values are collected during the withdrawal which can be associated with the withdrawn sample. The advantage of this configuration is that the second sensor unit 10′ can be disconnected from the bioreactor by the shut-off valves 4, 8, so that the sensors of the second sensor unit 10′ can be cleaned by blowing out the withdrawal line 3, and undesirable contact between the sensors and the organisms from the bioreactor can be avoided between the measurements.

FIG. 3 shows the bioreactor system, wherein the device for sampling, as a combination of the embodiments shown in FIGS. 1 and 2, comprises both the first sensor unit 10 and the second sensor unit 10′. In this way, the above-described advantages of both devices are combined. The device can accordingly be operated as described in connection with FIG. 1. However, additional advantages arise from the combination of the first sensor unit 10 and the second sensor unit 10′. For example, the second sensor unit 10′ can be configured to transmit a signal to the control unit 13 when the sample arrives at or passes the second sensor unit 10′ during the sampling process. The sampling process can then be terminated, for example, as soon as the sample arrives at the second sensor unit 10′, for example in that the second shut-off valve 8 is closed by the control unit. In this way, it is possible to control the volume of the withdrawn samples. For example, it can be ensured that every sample has the same volume or approximately the same volume. In a possible method in which the second shut-off valve is closed as soon as the sample reaches the second sensor unit 10′, the volume of the sample approximately corresponds to a line volume of the section of the withdrawal line 3 located between the T piece and the second sensor unit 10′. In some cases, such volume control is preferable to time control, in particular when the viscosity of the sample or the pressure conditions in the bioreactor system are not constant and, for example, change during the reaction or during the withdrawal. A combination of the described volume control with time control is likewise possible.

FIG. 4 shows the bioreactor system from FIG. 3, wherein a rinsing line 14 is provided in the withdrawal line 3 between the first shut-off valve 4 and the second sensor unit 10′. Sterile gas and/or liquids can be introduced into the flow in the withdrawal line 3 via the rinsing line 14, for example. This can be used, for example, for cleaning the withdrawal line 3 itself, in which case the second sensor unit 10′ does not have to be present in the sampling device. However, this can also be used to clean the sensors of the second sensor unit 10′ or be necessary for the use of the sensors of the second sensor unit 10′, for example when buffers that are used to reduce the impact on enzymatic sensors or to extend the service life thereof are fed into the withdrawal line via the rinsing line 14. In addition, it is possible for solutions for calibrating the sensors of the second sensor unit 10′ to be introduced into the flow. For introducing such solutions into the flow, these are supplied, for example, between two withdrawal processes via the rinsing line 14, and thereafter, with the first shut-off valve 4 open and the second shut-off valve 8 closed, the withdrawal line is blown out using sterile gas by way of the positive pressure source 5, so as to have no residue of the introduced solution, to the extent possible, present during the subsequent measurement which could adversely affect a measurement. The method steps described here which relate to the rinsing line can also be carried out with modified devices; for example, a first sensor unit 10 is also not necessary for this purpose.

FIG. 5 shows the bioreactor system as in FIG. 1, wherein additionally an optional rinsing line 14 is arranged at the withdrawal line, and a sterile coupling is provided between the first sensor unit 10 and the bioreactor 11. The sterile coupling comprises two coupling unions 111, 12.2, which can be connected to one another so as to establish a fluid connection, which is sealed to the outside, between two tubing or line ends at which the coupling unions are arranged. In the process, a first coupling union 12.1 is arranged in the rising line 1 on the side of the first sensor unit 10, and a second coupling union 12.2 is arranged in the rising line on the side of the bioreactor 11, so that the bioreactor 11, together with a portion of the rising line 1 and the second coupling union 12.2, can be disconnected from the remaining components of the sampling device. The shown embodiment comprising the sterile coupling is in particular of advantage when the sensor, or one of the sensors, of the first sensor unit 10″ is designed as a sensor coming in contact with media. Sensors coming in contact with the media are frequently not steam-sterilizable and can accordingly not be sterilized together with the bioreactor. The arrangement of the sterile coupling shown in the figure allows the section of the device comprising the sensor or sensors coming in contact with media to be sterilized separately, for example by gamma sterilization, plasma sterilization, or by means of ethylene oxide. So as to be able to sterilize the two sections of the device or of the bioreactor accordingly separately, the particular coupling unions connected to the sections are accordingly designed to be sterilizable. For example, the first coupling union 12.1 located on the side of the first sensor unit 10 can be gamma-sterilizable and made of plastic or steel, and the second coupling union 12.2 located on the side of the bioreactor 11 can be steam-sterilizable and made of plastic or metal.

FIG. 6 shows the bioreactor system, wherein the sterile coupling is arranged in the same location as in the example of FIG. 5, however, as is also shown in FIG. 2, only the second sensor unit 10′ is provided, instead of the first sensor unit 10. This configuration is particularly advantageous when the second sensor unit comprises sensors coming in contact with media. On the one hand, the rinsing line 14, as described above, allows the sensors to be cleaned and/or calibrated, and on the other hand, the sterile coupling makes it possible to separately sterilize sensors coming in contact with media. Otherwise, such a system is used as described in connection with FIG. 2.

As mentioned, the sterile coupling allows the sensors of the second sensor unit 10′ which come in contact with media to be sterilized separately from the bioreactor 11. The sterile filter 6 and the check valve 9 can then, for example, be gamma-sterilized together with the second sensor unit 10′, and thereafter can be connected to the autoclaved bioreactor 11. The shut-off valves 4, 8 can be configured as pinch valves, for example, and be installed after the sterilization.

FIG. 7 shows a combination of the embodiments from FIGS. 5 and 6. The sterile coupling is still arranged as the first component downstream of the bioreactor 11 in the rising line 1. In addition, the device comprises both the first sensor unit 10 and the second sensor unit 10′, and a rinsing line. In this way, both sensor units 10, 10′ can be sterilized separately from the bioreactor, for example when both sensor units comprise sensors that come in contact with media. Otherwise, the shown system is used as described in connection with FIG. 3, wherein additionally the above-described advantages of the rinsing line can be taken advantage of.

FIG. 8 shows the bioreactor system, wherein the sterile coupling is arranged in the withdrawal line 3, instead of in the rising line 1. In the process, the sterile coupling is positioned between the first shut-off valve 4 and the rinsing line 14. The bioreactor 11 can then be steam-sterilized, for example together with the check valve 9 and the sterile filter 6 as well as the rising line 1, the tap line 2, a portion of the withdrawal line 3, and the first coupling union 12.1, wherein the shut-off valves 4, 8 can be installed again after sterilization. The second sensor unit and the rinsing line 14, as well as the section of the withdrawal line 3 connected thereto and the second coupling union 12.2, can either be sterilized separately or be unsterile.

In other embodiments, such a configuration can also comprise a first sensor unit 10 and/or a sterile coupling as arranged in FIGS. 5 to 7.

FIG. 9 shows the bioreactor system, wherein the sensors and the sterile coupling are arranged as in FIG. 6, however a mixing chamber 16 is provided in the withdrawal line 3 between the second sensor unit and the first shut-off valve 4. A rinsing line 15 is connected to the mixing chamber 16 and furthermore comprises a mixing chamber drain 17 including a valve.

A sensor coming in contact with media, such as a pH sensor, can also be present in the mixing chamber 16.

During sampling by means of a system comprising such a mixing chamber 16, essentially the same steps are carried out as in the other described systems. The withdrawn sample first reaches the mixing chamber, before passing the second sensor unit 10′, and can then be left in the mixing chamber 16 for a certain period of time by accordingly briefly shutting off the pump 7 and/or by actuating valves. For this purpose, further valves can also be provided in possible embodiments. In the mixing chamber, the sample can be pretreated before being pumped further along the withdrawal line, so as to be measured by the second sensor unit 10′ and then be withdrawn or discarded.

A medium for diluting the sample may be added via the rinsing line 15. It is also possible, however, to add chemicals to the mixing chamber 16, for example, via the rinsing line 15, which enable a sensor measurement. For example, acids or lyes can be added, which shift the pH value of the sample in such a way that a measurement by way of a sensor of the second sensor unit 10′ is possible.

The mixing chamber can be emptied, for example, after use via the mixing chamber drain 17 and be cleaned by way of the rinsing line 15.

FIG. 10 shows, in table form, a possible sequence of steps for use of the bioreactor system according to the application, which are automatically triggered by the control unit, for example. In the process, steps S1 to S10 are carried out in the order listed here. A duration of each step is generally a few seconds. The status of the valves 4, 8 or of the pump 7 and the positive pressure source 5 during each step is indicated with “0” or “1” in the corresponding columns. In the process, “0” denotes “closed” or “off” and “1” denotes “open” or “on.” In a starting position S1, the valves 4, 8 are closed, and the pump 7 and the positive pressure source 5 are switched off. At an established point in time, for example at a point in time programmed in advance or in an event-controlled manner, triggered by a signal that is applied based on a value measured by a sensor 10, 10′, step S2 is initiated. In S2, pressure is applied to the tap line 2 by switching on the positive pressure source 5, while all other components remain in the “0” position. In S3, in addition to the positive pressure source 5 being switched on, the second shut-off valve 8 is opened, and the 1 is purged. Thereupon, in S4, the positive pressure source 5 is switched off so as to reduce the pressure in the rising line 1, while the second shut-off valve 8 remains open. In step S5, all valves 4, 8 are then closed again, and the positive pressure source 5 is left in the switched-off state, while the pump 7 is started up. In 56, positive pressure is generated in the rising line 1 in relation to the withdrawal line 3 by opening the first shut-off valve 4, while operating the pump at the same time. By subsequently additionally opening the second shut-off valve 8, in S7 the entire fluid path from the bioreactor 11 to the pump 7 is released, so that organisms can be withdrawn. The duration of this withdrawal process S7 varies as a function of the volume to be withdrawn. After the withdrawal has been completed, in S8 the second shut-off valve 8, and thus the rising line 1, are closed again so that the withdrawal line 3 can be blown out and cleaned. For this purpose, the positive pressure source 5 is additionally switched on. This step is carried out until it is ensured that the entire sample has reached the sample vessel. Thereafter, in S9, the rising line 1 is blown out by closing the first shut-off valve 4, and opening the second shut-off valve 8 again, while continuing to operate the positive pressure source 5. After having carried out steps 8 and 9, organisms that remained in the rising line 1 after the withdrawal process were recirculated to the reactor and organisms that remained in the withdrawal line were recirculated to the withdrawn sample, so that the process has no or a negligibly small dead volume. Thereafter, in the final position S10=S1 all elements can be returned to the “0” position until a new signal for sampling is issued.

In addition to the steps described here, it is possible, depending on the design and intended use of the design, as mentioned in connection with the other figures and in the description, to carry out additional steps, for example for forcing back the sample or for collecting measurement values by means of the first and/or the second sensor units, as well as for rinsing the second sensor unit, just to mention a few options. The listed possible steps thus do not represent an exhaustive list of the usage options of a device according to the application, or of a bioreactor system according to the application, but rather form a basis proceeding from which numerous further application options, which include further steps, for example, open up to a person skilled in the art.

### LIST OF REFERENCE NUMERALS

1 rising line
2 tap line
3 withdrawal line
4 first shut-off valve
5 positive pressure source
6 sterile filter
7 pump
8 second shut-off valve
9 check valve
10 first sensor unit
10′ second sensor unit
11 bioreactor
12.1 first coupling union
12.2 second coupling union
13 control unit
14 rinsing line
14 first rinsing line
15 second rinsing line
16 mixing chamber
17 mixing chamber drain

1. A device for sampling from a bioreactor, the device comprising:
a rising line connectable to a bioreactor;
a tap line; and
a withdrawal line;
wherein the rising line, the tap line, and the withdrawal line are connected to a T-like connection, wherein the withdrawal line includes a first shut-off valve; wherein the tap line is connected to a positive pressure source, wherein the withdrawal line is connected to a pump, and wherein the positive pressure source, the pump, and the first shut-off valve are automatically controlled via a control unit.

2. The device according to claim 1, wherein the tap line includes a sterile filter located between the positive pressure source and the T-like connection.

3. The device according to claim 1, wherein the rising line includes a second shut-off valve, which is automatically controlled via the control unit.

4. The device according to claim 1, wherein the tap line includes a check valve.

5. The device according to claim 1, wherein at least one of the rising line or the withdrawal line includes a sensor unit.

6. The device according to claim 1, wherein the rising line includes a first sensor unit, which is configured to transmit a measurement value to the control unit.

7. The device according to claim 6, wherein the first sensor unit comprises a contactless sensor.

8. The device according to claim 6, wherein the first sensor unit comprises a sensor that comes in contact with a media.

9. The device according to claim 1, wherein the withdrawal line comprises a second sensor unit.

10. The device according to claim 9, wherein the second sensor unit comprises a contactless sensor.

11. The device according to claim 9, wherein the second sensor unit comprises a sensor that comes in contact with a media.

12. The device according to claim 1, wherein at least one of the rising line and the withdrawal line includes a sterile coupling.

13. The device according to claim 1, wherein the withdrawal line includes a mixing chamber.

14. The device according to claim 1, wherein the control unit is configured to control at least the positive pressure source, the pump, and the first shut-off valve at a pre-designated time.

15. The device according to claim 6, wherein the control unit is configured to control at least the positive pressure source, the pump, and the first shut-off valve based on the measurement value of the first sensor unit.

16. A method of sampling a bioreactor, the method comprising:
providing a device at a bioreactor containing a sample, the device comprising:
a rising line;
a tap line; and
a withdrawal line;
wherein the rising line, the tap line, and the withdrawal line are connected to a T-like connection, wherein the withdrawal line includes a first shut-off valve, wherein the tap line is connected to a positive pressure source, wherein the withdrawal line is connected to a pump, and wherein the positive pressure source, the pump, and the first shut-off valve are automatically controlled via a control unit;
when the rising line is connected to the bioreactor, operating the pump;
operating the pump, with the first shut-off valve open and a second shut-off valve closed, for sampling;
applying a positive pressure to the tap line using the positive pressure source, while operating the pump, and with the first shut-off valve open and the second shut-off valve closed, for blowing out the withdrawal line; and
applying a second positive pressure using the positive pressure source, with the second shut-off valve open and the first shut-off valve closed, for blowing out the rising line.
